(19) United States
(12) Patent Application Publication
(10) Pub. No.: US 2002/0156046 A1
(43) Pub. Date: Oct. 24, 2002

(54) Sorbic acid product as addition to feedstuffs in agricultural livestock rearing

(76) Inventors: Nico N. Raczek, Kelkheim (DE); Hans-Ulrich Ter Meer, Frankfurt am Main (DE)

Correspondence Address: ProPat, L.L.C., 2912 Crosby Road, Charlotte, NC, 28211-2815, US

(21) Appl. No.: 10/056,328

(22) Filed: Jan. 24, 2002

(30) Foreign Application Priority Data
Feb. 2, 2001 (DE) 101 05 305.3

(51) Int. Cl. A61K 31/715; A61K 31/718; A61K 31/19; A61K 31/7016

(52) U.S. Cl. 514/53; 514/54; 514/60; 514/557

(57) ABSTRACT

The present invention relates to a product for use in animal feedstuffs. The product comprises sorbic acid and at least one NDS. The invention further relates to the use of the product on its own in feedstuffs or mixed with other feedstuff additives for improving the hygienic status of the feed and for improving performance in agricultural livestock rearing.

### BACKGROUND OF THE INVENTION

The invention relates to a product which comprises sorbic acid and at least one nondigestible saccharide (−ND3) and can be used on its own in feedstuffs or mixed with other feedstuff additives in agricultural livestock rearing.

Antibiotics are frequently used to improve performance in the animal feed sector. The use of antibiotics in this sector is suspected of being responsible for the dangers derived from resistant bacteria, which may also endanger human health in the long term. It is therefore necessary to look for products about which there are fewer health doubts for this purpose of use. Thus, in other sectors too there is increasing replacement of substances about which there are physiological and epidemiological health doubts or else which are harmful for the environment, such as, for example, antibiotics, formaldehyde-emitting materials, halogenated substances and many others, by materials about which there are fewer doubts, for example in human foods, feedstuffs, domestic animal feed, silages, pomace, or other waste materials from the food industry. The purpose of these materials is, on the one hand, to maintain the value of the actual product. However, on the other hand, it is also intended to improve the hygienic conditions thereof and achieve a longer shelf life.

It is known that sorbic acid can be employed for preserving foodstuffs. Sorbic acid (trans, trans-2,4-hexadiene acid) is a colorless solid compound which dissolves only slightly in cold water and is used around the world as preservative. The principle of action is determined by sorbic acid in undissociated form. Sorbic acid therefore displays its best effect in the acidic pH range. Sorbic acid and its salts have a very good microbiostatic, antimycotic action. At the same time, as unsaturated fatty acid, sorbic acid is virtually nontoxic, which is proven by very extensive data and by the decades of use of this in the human food sector, in animal feeds, inter alia.

Besides sorbic acid, other organic acids have also been employed for some years for preserving foodstuffs and for improving feed hygiene. The hygienic quality in particular of feed for young animals must meet special requirements. This is why some organic acids are approved without a limitation on the maximum amount, on the basis of the national legal provisions concerning foodstuffs.

There have already been various investigations of the use of oligosaccharides for increasing performance in agricultural livestock rearing, and beneficial effects on weight gain and feed conversion have been found (Beck, M. et al., meeting proceedings of the 6th Pig and Poultry Nutrition meeting, 2000, pages 190-192; Bolduan et al., DGS MAGAZIN, week 48/99, pages 39-40).

The overall disadvantage of previously disclosed acid-based additions to feedstuffs has been that they are relatively unstable. The acids suitable for acidification are in some cases volatile, have unpleasant odors and, in addition, have corrosive effects. The performance-improving effects which can be achieved with them are associated with considerable disadvantages in handling. However, these acids have corrosive effects and, because of their volatility, in some cases cause an odor nuisance and require special care in handling if the risk of intake by inhalation, which is undesirable from the health and safety viewpoint, is to be avoided. The object accordingly was to provide an addition which is easy to handle and maximally improves performance and does not have these disadvantages.

### BRIEF DESCRIPTION OF THE INVENTION

This object is achieved by a product (composition) which comprises sorbic acid and at least one NDS.

### DETAILED DESCRIPTION OF THE INVENTION

NDS means nondigestible materials which, in the animal body, remain undigested in the sections of the digestive tract which are not naturally colonized by microorganisms but which can be broken down in the parts of the digestive tract which are colonized by microorganisms and thus specifically increase the growth and/or the activity of one or more groups of microorganisms in the intestinal tract to an extent such that beneficial effects on the animal body result. The NDS used according to the invention have chain lengths of 2-80, preferably 2-20. The term NDS also includes various polysaccharides such as resistant starches, β-glucans, hemicelluloses, pectins, mannans such as galactomannans, glucomannans, galactoglucomannans, xylans such as arabinoxylans, galactans such as arabinogalactans and vegetable gums, and the latter may likewise promote the growth of microorganisms in the intestinal tract. In place of pure NDS as defined above, it is also possible to use feedstuff components in which the content of these NDSs are at least 25% by weight, and, in particular, products in which these NDSs are concentrated to more than 25% by weight. Examples thereof are yeast extracts, constituents of microorganism cells, bran (fractions), residues from pressing in the processing of fruits and vegetables or pressed cakes from oil extraction and fiber extracts from the flesh of the fruit of the locust bean tree (Ceratonia siliqua).

Examples of NDS which can be employed according to the invention are:

fructooligosaccharides which are produced, for example, from sucrose;

Inulin which has been obtained, for example, from chicory, Jerusalem artichokes, onions, leaks, artichokes, bananas and wheat;

oligofructoses which have been produced, for example, by enzymatic hydrolysis from inulin;

galactooligosaccharides, transgalactosyloligosaccharides which can be produced from lactose;

oligosaccharide mixtures, for example from soybeans;

xylooligosaccharides, for example from corn cobs;

lactulose from lactose;

lactosucrose from lactose and sucrose;

glucooligosaccharides, isomaltooligosaccharides from sucrose and/or maltose;

gentiooligosaccharides from glucose syrup;

starch hydrolysates which contain isomaltooligosaccharides and/or maltooligosaccharides.

The abovementioned NDS also include feedstuff constituents if the proportions of NDS they contain are at least 25% by weight.

The NDS are present in the products of the invention in amounts of 90-10% by weight, preferably 70-30% by weight.

The product of the invention contains 10-90% by weight, preferably 30-70% by weight, sorbic acid.

Examples of suitable animal feedstuffs are green fodder, silages, dried green fodder, roots, tubers, fleshy fruits, grains and seeds, brewer's grains, pomace, brewer's yeast, distillation residues, milling byproducts, byproducts of the production of sugar and starch and oil production and various food wastes. Feedstuffs of these types may be mixed with certain feedstuff additives (e.g. antioxidants) or mixtures of various substances (e.g. mineral mixtures, vitamin mixtures) for improvement. Specific feedstuffs are also adapted for particular species and their stage of development. This is the case, for example, in piglet rearing. Prestarter and starter feeds are used here. The product of the invention can be added to the animal feedstuff directly or else mixed with other feedstuff additives or else be added via premixes to the actual feedstuff. The product can be admixed dry with the feed, be added before further processing (e.g. extrusion) or be metered in and dispersed in the mixture. An additional possibility is to add the individual ingredients of the product separately to the feedstuff. It is expedient to use for this purpose product concentrations between 0.2 and 5.0% by weight (based on the feed), preferably 1.0-3.0% by weight.

The product of the invention is produced by, for example, mechanically mixing the sorbic acid and the solid pasty NDS. It is likewise possible to provide sorbic acid and the NDS separately—especially when the NDS are in the form of syrupy or liquid preparations—to mix these successively and uniformly into the feed.

The product can be added as sole additive to animal feedstuffs, for example for rearing calves or lambs, particularly preferably to prestarter and starter feeds for piglets, or be used mixed with other feed additives for these stocks.

Surprisingly, the products of the invention do not show the disadvantages described above. On the contrary, the products show good handling properties. The breakdown of these NDS, which are not directly digestible, to short-chain acids results in an improvement in the hygienic conditions in the animals' intestinal tract and a repression of the growth of unwanted microbes. In addition, surprisingly, a beneficial effect on the growth performance of young stock can also be found.

The selective addition to the animal feed provides the target organisms with an advantage over all other bacteria. The target organisms, which are often lactic acid-producing species, have their growth significantly promoted and influence the health of the stock in a beneficial manner.

It has surprisingly been found that a marked improvement in performance in relation to growth rate and feed conversion can be achieved by adding even small amounts of products of the invention in piglet rearing. To ensure a significant activity in improving performance, the minimum amount of sorbic acid is expediently 0.1% by weight, preferably at least 0.5% by weight, and the minimum amount of NDS is expediently 0.1% by weight, preferably 0.3% by weight, based on the feed. Feedstuffs having the product of the invention are moreover suitable as milk replacers for the early weaning of lambs or calves.

The presence of the NDS additionally promotes the growth of many probiotic microorganisms. It is an additional advantage for these organisms that the breakdown of the NDS is extended. It may therefore be advantageous also to add probiotics to the products of the invention.

Probiotics are intended to mean viable forms of microorganisms or spores which can be supplied to the stock continuously with the feed. They comprise selected strains of yeasts or lactic bacteria (morphologically variable gram-positive, non-motile and catalase-negative bacteria, such as Streptococcacaeae, including bacteria of the genus Enterococcus, Lactobacillaceae, Bacillaceae or Actinomycetaceae). They are, moreover, strains which are particularly acid-resistant. In the case of spore formers, the spores are used as feed additive.

The following microorganisms or combinations thereof are preferred:

Bacillus cereus (especially Bacillus cereus var. toyoi)

Bacillus clausii.

Bacillus licheniformis

Bacillus subtilis

Bifidobacterium bifidum

Bifidobacterium breve

Bifidobacterium infantis

Bifidobacterium lactis

Bifidobacterium longum

Bifidobacterium adolescentis

Enterococcus faecium

Enterococcus mundtii

Lactobacillus acidophilus

Lactobacillus amylovorus

Lactobacillus bulgaricus (especially Lactobacillus delbrueckii subsp. bulgaricus)

Lactobacillus casei

Lactobacillus crispatus

Lactobacillus farciminis

Lactobacillus gallinarum

Lactobacillus gasseri

Lactobacillus johnsonii

Lactobacillus paracasei

Lactobacillus plantarum

Lactobacillus reuteri

Lactobacillus rhamnosus

Lactobacillus salivarius

Pediococcus acidilactici

Saccharomyces cerevisiae

Streptococcus infantarius

Streptococcus thermophilus (especially Streptococcus salivarius subsp. thermophilus).

The microorganisms or combinations thereof can be added to the products of the invention in amounts which correspond to 10⁷ to 10¹⁰, preferably 0.1-50×10⁹ CFU (colony-forming units) of, in particular, viable microorganisms per kg of feedstuff.

If the product of the invention contains probiotics, it can be produced by uniform mixing of a spore-containing microorganism culture, where appropriate immobilized on a carrier or encapsulated, and the other ingredients of the product. In the case of live cultures, the microorganisms, where appropriate immobilized on a carrier or encapsulated, and the other ingredients are provided separately and are mixed successively and uniformly into the feedstuff. Microorganisms can be immobilized, for example, by spraying culture solutions onto appropriate carriers. Examples suitable for the encapsulation are gelatine, polysaccharides or else the NDS employed according to the invention.

The invention is illustrated below by means of examples:

In order to investigate the growth-stabilizing activity of sorbic acid in combination with an NDS or with sorbic acid, an NDS and an additionally added probiotic, trials were carried out on piglets which were 28 days old and had an average weight of 8.9 kg at the start of the trial. At the start of the trial (3 days after rehousing), the piglets were assigned at random, taking account of parentage, weight and sex, to 5 trial groups each of 12 animals. In each case 8 animals from a litter formed a block in which the treatments were assigned to the animals at random. The trial design thus corresponded to a completely randomized block system. The piglets were held in single pens in a flat-deck system.

The following division into groups was undertaken for investigations of the nutritional activity of the various groups of active substances:

| Group | Addition to each feed |
|---|---|
| A | Negative control, no supplement |
| B | 0.875% sorbic acid + 0.03 g/kg Oralin ® (corresponds to |
| | about 1.05 × 10⁹ CFU/kg) |
| C | 0.875% sorbic acid + 0.5% FOS (fructooligosaccharide) |
| D | 0.03 g/kg Oralin ® + 0.5% FOS |
| E | 0.875% sorbic acid + 0.03 g/kg Oralin ® + 0.5% FOS |

The FOS (fructooligosaccharide) used in the trials is a prebiotic standardized by enzymatic conversion of beet sugar and having the structure α-D-Glu[-(1→2)-β-D-Fru]ₙ, representing 37±6% with n=2, 53±6, 53±6% with n=3 and 10±6% with n=4 (proprietary name: “Anta-Sweet 295”, marketed by Dr. Eckel GmbH, 56651 Niederzissen).

The probiotic “Oralin®” (marketed by Bayer Chemie Service GmbH, 51063 Cologne) which was used contains Enterococcus faecium (DSM 10663; NCIMB 10415) microbes in an activity of 3.5×10¹⁰ CFU/g, which was employed in the form of microgranules.

These additives were mixed into the basic feed by varying the proportions of corn starch and soybean oil so as to be protein- and energy-equivalent, so that the compositions of the trial feeds were nutritionally equivalent. Table 1 indicates the composition of the basic feed mixes employed. Two different types of feed were used consecutively in this trial. In the first 21-day section up to a weight of about 18 kg, a so-called prestarter corresponding to a standard supplementary feed for piglets was employed, and in the second 17-day section up to the end of the trial a so-called starter feed for piglets (standard: complete piglet feed) was employed.

The feed mixtures were provided in the form of meal ad libitum. The feed was provided each day, and unconsumed food was reweighed twice a week. Drinking water was available without restriction from drinkers. The trail criteria recorded were the weight gain, daily growth rate, feed consumption and feed conversion (feed consumption in g/g of weight gain). Mathematical statistical analysis of the trial data was carried out for the entire trial period and for each section of the trial separately by analysis of variance.

The results obtained in the complete rearing period have been summarized in table 2. Their final weights were 5.5% higher with combined addition of sorbic acid and FOS and 4.9% higher with addition of sorbic acid and Oralin compared with the animals without these additions in their feed. The final weights were not increased on administration of Oralin together with FOS, while the sorbic acid+Oralin+FOS combination increased them by 5.7%. The effect according to the invention on the weight gain of the piglets was thus shown on addition of NDS with sorbic acid or the addition of the NDS/sorbic acid combination with a probiotic. The daily growth rate corresponded with this; the growth rates were increased by 6.3% and 5.9% on combined use of respectively sorbic acid+Oralin and sorbic acid+FOS. Without sorbic acid in the feed there was no growth-promoting effect when Oralin+FOS were present together in the feed. By contrast, the sorbic acid+Oralin+FOS combination greatly increased growth by 7.7% according to the invention. The use of sorbic acid together with Oralin or FOS (group B or C) significantly improved the feed conversion to 1.59 and 1.57 g/g respectively (respectively 6.0% and 7.1% compared with the negative control). The feed conversion was reduced only very slightly to 1.66 g/g (1.8%) on simultaneous feeding of Oralin+FOS (group D). The sorbic acid+Oralin+FOS combination (group E) resulted in a feed conversion of 1.56 g/g, which was significantly more favorable, by 7.7%, than in the negative control and thus showed the best result of all the variants tested.

Overall assessment of the trials reveals that only slight or no growth-stabilizing effects are to be found with combined use of FOS and Oralin, that is to say an NDS and probiotic. Considerably higher growth rates and a better feed conversion of the stock are achieved with a combination of sorbic acid with FOS (NDS). This also applies to the combination of the invention, of FOS (NDS) and Oralin (as probiotic).

TABLE 1
Composition of the feedstuffs used (%)

| Component | Prestarter | Starter |
|---|---|---|
| Corn | 30.0 | 21.25 |
| Wheat | 29.03 | 20.00 |
| Barley | — | 25.0 |
| Extracted soybean meal | 15.23 | 27.34 |
| Skimmed milk powder | 14.00 | — |
| Potato protein powder | 5.00 | — |
| Corn starch | 1.70 | 1.70 |
| Soybean oil | 1.53 | 1.11 |
| L-lysine · HCl | 0.05 | 0.23 |
| DL-methionine | 0.03 | 0.04 |
| L-threonine | — | 0.04 |
| Mineral feed, vit. | 3.43 | 3.29 |
| Nutrient analysis (g/kg) | | |
| Crude protein | 211 | 186 |
| ME, MJ/kg | 13.95 | 13.2 |
| Lysine | 12.5 | 11.0 |
| Methionine + cysteine | 7.5 | 6.6 |
| Threonine | 8.6 | 7.3 |
| Tryptophan | 2.5 | 2.2 |
| Calcium | 9.8 | 8.5 |
| Phosphorus | 7.3 | 6.0 |
| Sodium | 2.3 | 1.8 |

TABLE 2
Growth parameters of piglets on use of sorbic acid, a pro- or prebiotic
and combinations of these additions

| Group | A | B | C | D | E |
|---|---|---|---|---|---|
| Sorbic acid, % | — | 0.875 | 0.875 | — | 0.875 |
| Probiotic | — | Oralin ® | — | Oralin ® | Oralin ® |
| Prebiotic | — | — | FOS | FOS | FOS |
| Initial weight, kg | 8.80 | 8.97 | 9.19 | 8.89 | 8.89 |
| | ±1.18 | ±1.37 | ±1.32 | ±1.05 | ±1.26 |
| Final weight, kg | 29.94 | 31.42 | 31.58 | 30.09 | 31.64 |
| | ±3.65 | ±3.42 | ±3.64 | ±3.05 | ±4.73 |
| relative | 100 | 104.9 | 105.5 | 100.5 | 105.7 |
| Growth rate, g/d | 556 | 591 | 589 | 558 | 599 |
| | ±77 | ±64 | ±71 | ±82 | ±101 |
| relative | 100 | 106.3 | 105.9 | 100.4 | 107.7 |
| Feed | 939 | 937 | 927 | 924 | 933 |
| consumption, g/d | ±115 | ±115 | ±112 | ±149 | ±158 |
| relative | 100 | 99.8 | 98.7 | 98.4 | 99.4 |
| Feed conversion | 1.69^{a} | 1.59^{bc} | 1.57^{bc} | 1.66^{ab} | 1.56^{c} |
| per g gain, g | ±0.05 | 0.08 | ±0.05 | ±0.11 | ±0.07 |
| relative | 100 | 94.0 | 92.9 | 98.2 | 92.3 |

1. A product which comprises sorbic acid and at least one NDS (nondigestible saccharide).

2. A product as claimed in claim 1, wherein the product contains at least 10% by weight sorbic acid.

3. A product as claimed in claim 1, which comprises from 10 to 90% by weight sorbic acid (based on the product).

4. A product as claimed in claim 1, which comprises from 90 to 10% by weight, (based on the product) of NDS.

5. A product as claimed in claim 1, wherein the NDS is selected from one or more of the following substances
Fructooligosaccharides
Inulin
Oligofructoses
Galactooligosaccharides
Transgalactosyloligosaccharides
Oligosaccharide mixtures
Xylooligosaccharides
Lactulose
Lactosucrose
Glucooligosaccharides
Isomaltooligosaccharides
Gentiooligosaccharides
Starch hydrolysates which contain isomaltooligosaccharides and/or maltooligosaccharides.

6. A product as claimed in claim 1, which additionally comprises at least one probiotic.

7. A product as claimed in claim 6, wherein the content of probiotic or probiotics is from 10⁷ to 10¹⁰ CFU (colony-forming unit).

8. A feedstuff which comprises a product as claimed in claim 1.

9. An addition to feedstuffs which comprises a product as claimed in claim 1.

10. A feedstuff as claimed in claim 8, which comprises from 0.2 to 5.0% by weight (based on the feedstuff) of the product.

11. The method of using the product as claimed in claim 1 as addition to animal feeds or feedstuffs, which method comprises adding a product as claimed in claim 1 to animal feeds or feedstuffs.

12. The method as claimed in claim 11 in pig rearing.

13. The method as claimed in claim 11 in cattle rearing.

14. The method as claimed in claim 11 in lamb rearing.

15. The method as claimed in claim 11 in poultry rearing.
